(19) United States
(12) Patent Application Publication
(10) Pub. No.: US 2007/0166386 A1
(43) Pub. Date: Jul. 19, 2007

(54) Nanoparticle formation of pharmaceutical ingredients

(76) Inventors: Vanessa I. Chinea, Aguadilla, PR (US); Kevin M. Kane, Ft. Lauderdale, FL (US); Isaac Farr, Corvallis, OR (US); Iddys D. Figueroa, Aguadilla, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/332,131

(22) Filed: Jan. 13, 2006

(51) Int. Cl. A61K 9/14

(52) U.S. Cl. 424/489; 977/906

(57) ABSTRACT

A pharmaceutical ingredient is dissolved within a solvent. The solvent is evaporated until nanoparticles of the pharmaceutical ingredient are at least partially formed without employing a substrate for them. A portion of the solvent remains, within which the nanoparticles are located.

### BACKGROUND

Oral administration of pharmaceuticals is one of the most widely used methods for providing therapy to treat a variety of illnesses. Many medications are orally administrated to a person in a dosage form, such as a tablet, capsule, or liquid. Such medications can be administered buccally, sublingually, or swallowed for release into the digestive tract.

In order for a drug to achieve its desired result, it typically has to be delivered to a biological site of interest. Most drugs in use today are solid ingestibles. For these drugs to be absorbed into the bloodstream and transported to a biological site of interest, they usually have to first be dissolved and then permeate the intestinal walls. The drugs also should avoid first-pass metabolism, which occurs when the drugs are removed from the bloodstream as they pass through the liver.

The preparation of small particles can increase the solubility and potentially the bioavailability of a selected drug candidate. Solubility may be modified by physically grinding a drug to yield micron size and smaller particles. However, this mechanical approach can cause chemical or physical degradation of the drug, by shearing and heat stress. Furthermore, particles less than five microns in size tend to agglomerate, which counters the benefits of micronization.

Spray-drying and freeze-drying may also be used to generate small particles to increase drug dissolution rates, and thus bioavailability. However, agglomeration remains a problem with these approaches. Other approaches to increase the solubility and thus the bioavailability of drugs likewise have difficulties associated with them. For these and other reasons, therefore, there is a need for the present invention.

### BRIEF DESCRIPTION OF THE DRAWINGS

The drawings referenced herein form a part of the specification. Features shown in the drawing are meant as illustrative of only some embodiments of the invention, and not of all embodiments of the invention, unless otherwise explicitly indicated.

FIG. 1 is a flowchart of a method for forming nanoparticles of a pharmaceutical ingredient, according to an embodiment of the invention.

FIGS. 2A, 2B, 2C, and 2D are diagrams illustratively depicting different parts of the method of FIG. 1, according to varying embodiments of the invention.

FIG. 3 is a flowchart of another method for forming nanoparticles of a pharmaceutical ingredient, according to another embodiment of the invention.

FIGS. 4A and 4B are diagrams illustratively depicting performance of most of the method of FIG. 3, according to different embodiments of the invention.

### DETAILED DESCRIPTION OF THE DRAWINGS

In the following detailed description of exemplary embodiments of the invention, reference is made to the accompanying drawings that form a part hereof, and in which is shown by way of illustration specific exemplary embodiments in which the invention may be practiced. These embodiments are described in sufficient detail to enable those skilled in the art to practice the invention. Other embodiments may be utilized, and logical, mechanical, and other changes may be made without departing from the spirit or scope of the present invention. The following detailed description is, therefore, not to be taken in a limiting sense, and the scope of the present invention is defined only by the appended claims.

### First Embodiment

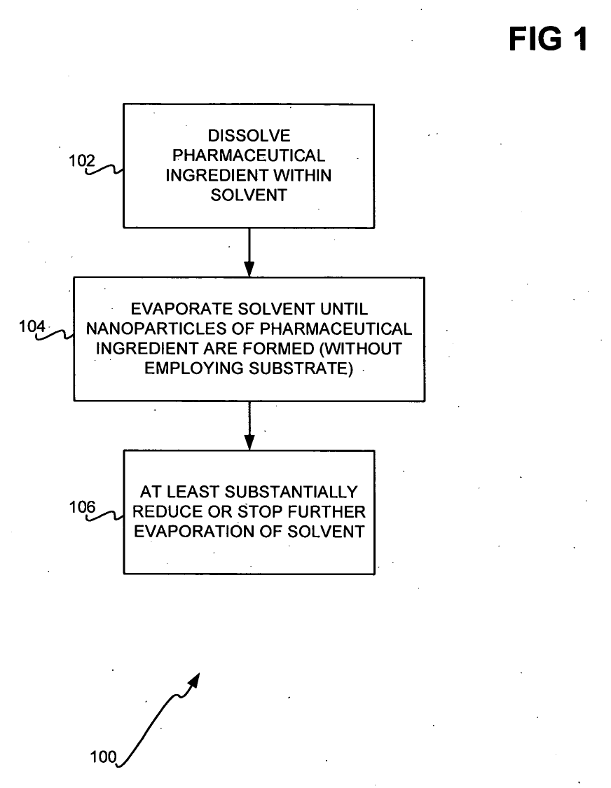

FIG. 1 shows a method 100 for forming nanoparticles of a pharmaceutical ingredient, according to an embodiment of the invention. The pharmaceutical ingredient may be an active pharmaceutical ingredient, such as glyburide, prednisolone, or indomethacin, among other types of active pharmaceutical ingredients. Other types of pharmaceutical ingredients, for instance, include betamethasone acetate, triamcinolone acetonide, piroxicam, glimepiride, glipizide, and digoxin.

First, the pharmaceutical ingredient is dissolved within a solvent (102). For instance, the solvent may be a binary solvent, such as ethanol:chloroform having a proportion of 80% ethanol to 20% chloroform by volume (i.e., 80% of the volume of the solvent is ethanol, and 20% of the volume is chloroform). The resulting solution may be five milligrams of pharmaceutical ingredient per milliliter (mL) of solvent solution. Other solvent combinations have also been proven to result in nanoparticle formation. These include 80% ethanol by volume and 20% water by volume; 80% methanol by volume and 20% water by volume; and, 80% acetone by volume and 20% water by volume. It is noted that the terminology “solvent” as used herein is inclusive of the plural “solvents,” when, for instance, a binary solvent, or another type of multiple solvent, like a ternary solvent, is used.

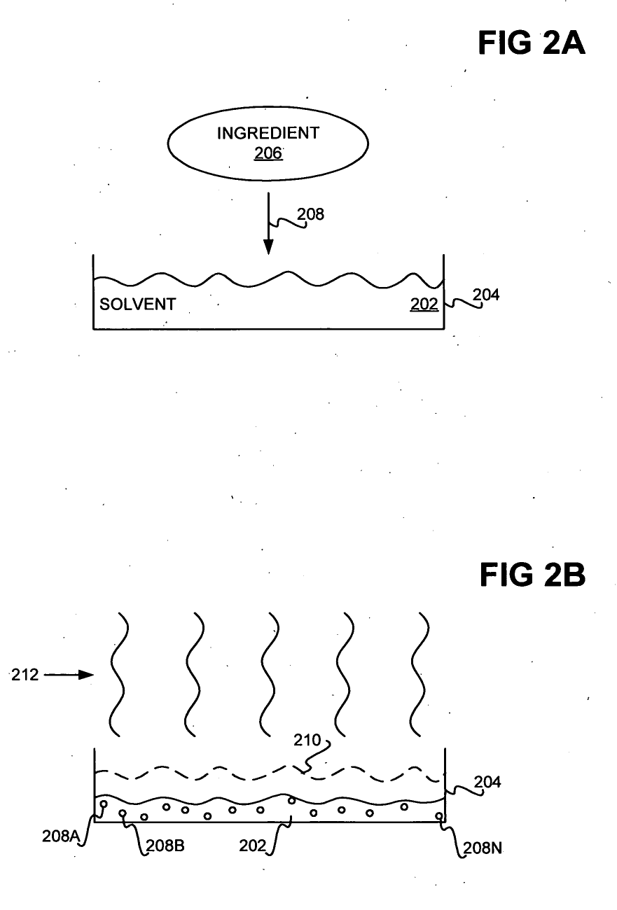

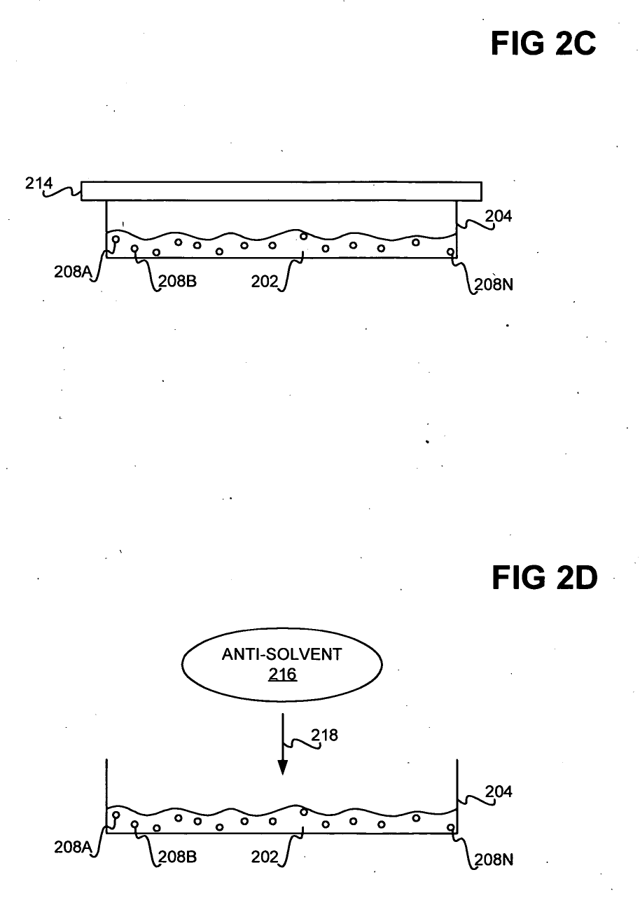

FIG. 2A illustratively depicts performance of part 102 of the method 100, according to an embodiment of the invention. A solvent 202 is situated within a container 204. A pharmaceutical ingredient 206 is added to the solvent 202, as indicated by the arrow 208, and dissolved within the solvent 202. It is noted that there is no substrate present within FIG. 2A, which, for instance, differentiates one embodiment of the invention from that which is disclosed in the U.S. patent application Ser. No. ______ [attorney docket no. 200501062-1].

Referring back to FIG. 1, the solvent, within which the pharmaceutical ingredient has been dissolved (i.e., resulting in a solution of the ingredient dissolved within the solvent), is evaporated until nanoparticles of the pharmaceutical ingredient are formed (104). That is, it has been discovered that evaporating such a solvent within which a pharmaceutical ingredient has been dissolved results in formation of nanoparticles of the pharmaceutical ingredient. The particles of the pharmaceutical ingredient are nanoparticles in that they can be substantially round and/or spherical in shape, and further have dimensions measurable in nanometers. These nanoparticles may also be referred to as nanopearls.

Evaporation occurs at a rate that allows formation of the nanoparticles, without other larger particles and polymorphs of the drug in question being produced. This evaporation rate may be experimentally and empirically determined, but in one embodiment can be one mL per minute. In another embodiment, this evaporation rate may be one mL per three-to-five minutes, or 0.2 mL per minute.

The formation of nanoparticles of the pharmaceutical ingredient within the solvent occurs without employing a substrate. That is, for example, the solution of the solvent and the pharmaceutical ingredient is not placed on a substrate to promote or assist formation of the nanoparticles within the solvent. Rather, no substrate is used. As such, this embodiment of the invention is in contradistinction to that which is disclosed in the U.S. patent application Ser. No. ______ [attorney docket no. 200501062-1], in which a substrate was thought to be required in order to form-nanoparticles.

FIG. 2B illustratively depicts performance of part 104 of the method 100, according to an embodiment of the invention. The solvent 202 within the container 204 has been evaporated, as indicated by the vapor 212, so that the original amount of the solvent 202 present in FIG. 2A, as indicated by the dotted line 210 in FIG. 2B, has been reduced in volume to the amount thereof shown in FIG. 2B. This controlled evaporation of the solvent 202 results in a number of nanoparticles 208A, 208B, . . . , 208N, collectively referred to as the nanoparticles 208, of the pharmaceutical ingredient 206 dissolved within the solvent 202 as has been depicted in FIG. 2A.

Referring back to FIG. 1, the method 100 concludes by at least substantially reducing or stopping further evaporation of the solvent (106), once the nanoparticles of the pharmaceutical ingredient have formed therein. In one embodiment, reduction or stoppage of further evaporation of the solvent is achieved by simply sealing the container within which the solvent is situated. In another embodiment, an anti-solvent is added to the solvent, to reduce or stop further evaporation of the solvent. The anti-solvent may be water, ethanol-water mixtures, alkanes such as pentane, hexane, heptane, or octane; alkenes such as pentene, hexene, heptene, or octene; cycloalkanes such as cyclohexane or cyclooctane; supercritical fluids such as carbon dioxide; or, ethers such as diethyl ether, among other types of anti-solvents. The anti-solvent may also be referred to as a non-solvent.

FIGS. 2C and 2D illustratively depict performance of part 106 of the method 100, according to different embodiments of the invention. In FIG. 2C, the container 204, containing the solvent 202 within which the nanoparticles 208 of the pharmaceutical ingredient 206 have been formed, is sealed with a lid or cap 214. The cap 214 prevents further evaporation of the solvent 202. In FIG. 2D, by comparison, instead of a cap sealing the container 204 to reduce or stop further evaporation of the solvent 202, an anti-solvent 216 is added to the solution of the solvent 202 and the nanoparticles 208, as indicated by the arrow 218. The addition of the anti-solvent also reduces or stops further evaporation of the solvent 202.

The method 100 of FIG. 1 that has been described, and illustratively depicted in FIGS. 2A, 2B, 2C, and 2D, thus relies upon evaporation of a solvent within which a pharmaceutical ingredient has been dissolved to achieve nanoparticle formation of the pharmaceutical ingredient. The method 100 further does not employ a substrate to assist or promote such nanoparticle formation, in contradistinction with earlier approaches to form nanoparticles, in which it was thought that the substrate played an important if not necessary role to nanoparticle formation. Sealing of the solvent or addition of an anti-solvent is used in the method 100 to stop or at least substantially reduce further evaporation of the solvent, once desired nanoparticle formation has occurred.

### Second Embodiment

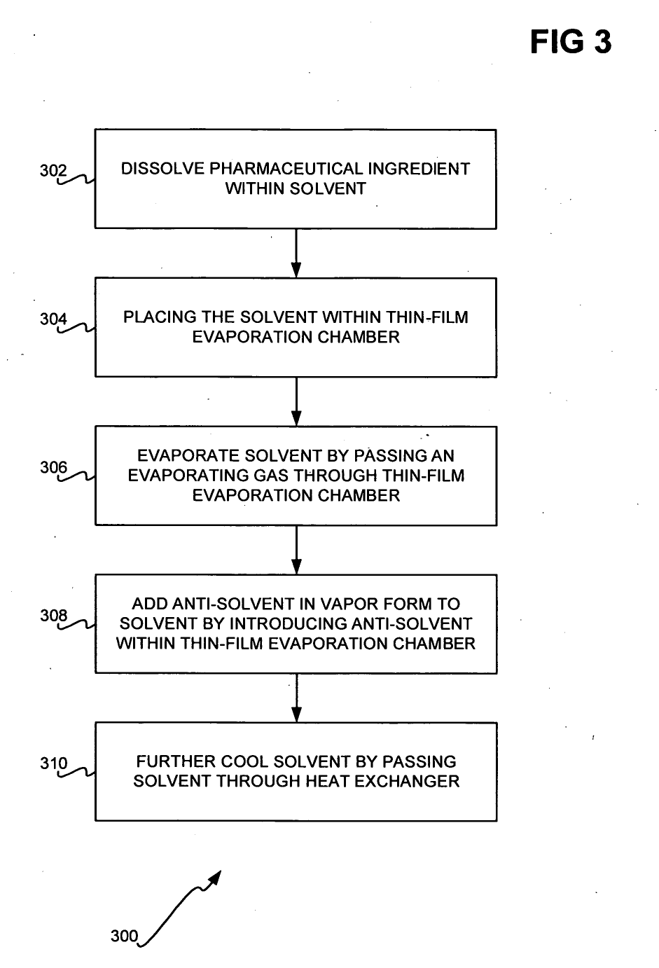

FIG. 3 shows a method 300 for forming nanoparticles of a pharmaceutical ingredient, according to another embodiment of the invention. As in the method 100 of FIG. 1, the pharmaceutical ingredient may be an active pharmaceutical ingredient, such as glyburide, prednisolone, or indomethacin, among other types of active pharmaceutical ingredients. First, the pharmaceutical ingredient is dissolved within a solvent (302), as before. For instance, the solvent may be a binary solvent, such as ethanol:chloroform having a proportion of 80% ethanol to 20% chloroform by volume. The solvent may also be a single solvent such as methanol, ethanol, isopropanol, acetone, or acetonitrile.

Next, the solvent, within which the pharmaceutical ingredient has been dissolved, is placed within a thin-film evaporation chamber (304). It is noted here that any number of different configurations of thin-film evaporators may be used, as can be appreciated by those of ordinary skill within the art. Placement of the solvent within the thin-film evaporation chamber provides a manner by which the solvent can be evaporated and an anti-solvent can be added to the solution of the solvent and the pharmaceutical ingredient. In this embodiment, evaporation of the solvent also contributes to formation of nanoparticles of the pharmaceutical ingredient. However, the addition of the anti-solvent, via condensation from vapor or via direct anti-solvent addition onto the solvent, also contributes to formation of the nanoparticles of the pharmaceutical ingredient.

The solvent is evaporated by passing an evaporating gas through the thin-film evaporation chamber (306). In one embodiment, the evaporating gas can be compressed air with varying levels of water content, an inert gas like helium, argon, nitrogen, carbon dioxide, and so on, or another type of evaporating gas. Evaporation of the solvent results in a reduction of the volume of the solvent, as well as a decrease in temperature of the solvent. Additional steps or acts may be performed to further reduce the volume and/or decrease the temperature of the solvent, one of which is particularly described later in the detailed description.

Anti-solvent is also added in vapor form to the solvent by introducing the anti-solvent vapor within the thin-film evaporation chamber (308). The anti-solvent may as before be water, or another type of anti-solvent. The anti-solvent vapor may be introduced within the thin-film evaporation chamber by passing it through the chamber, similar to the evaporating gas. There is a short diffusion distance within the thin film of the solvent, which enables the anti-solvent to quickly establish a homogeneous concentration within the solution of the solvent and the pharmaceutical ingredient. Therefore, localized high anti-solvent concentrations within the solvent are avoided and which would otherwise result in a heterogeneous environment of solvent and anti-solvent.

Upon evaporation of the solvent and condensation (i.e., addition or precipitation) of the anti-solvent, the resulting final solution of the solvent, anti-solvent, and the pharmaceutical ingredient has at least a substantially optimal solvent-to-anti-solvent ratio, temperature, and degree of supersaturation of the pharmaceutical ingredient. These optimal conditions result in precipitated nanoparticles of the pharmaceutical ingredient forming within the solvent. Thus, both evaporation of the solvent and addition of the anti-solvent contribute to nanoparticle formation, as has been noted above.

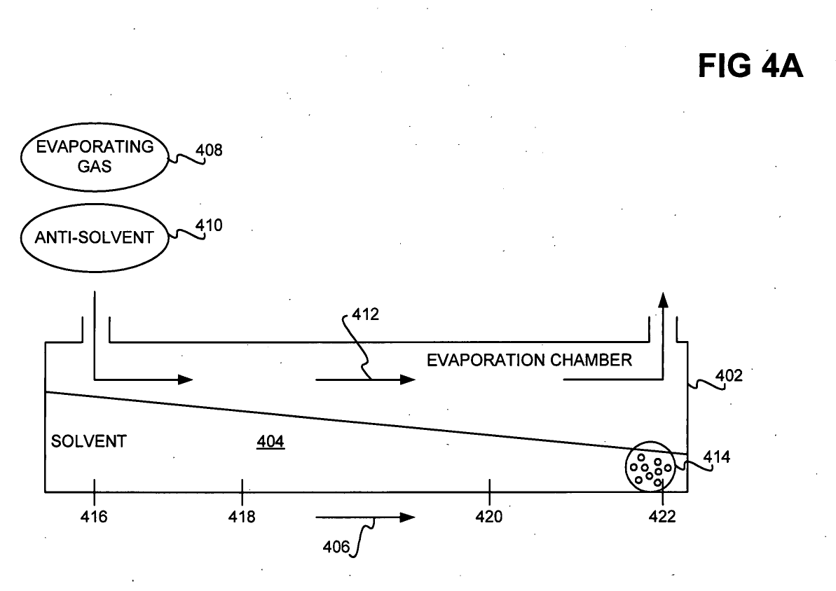

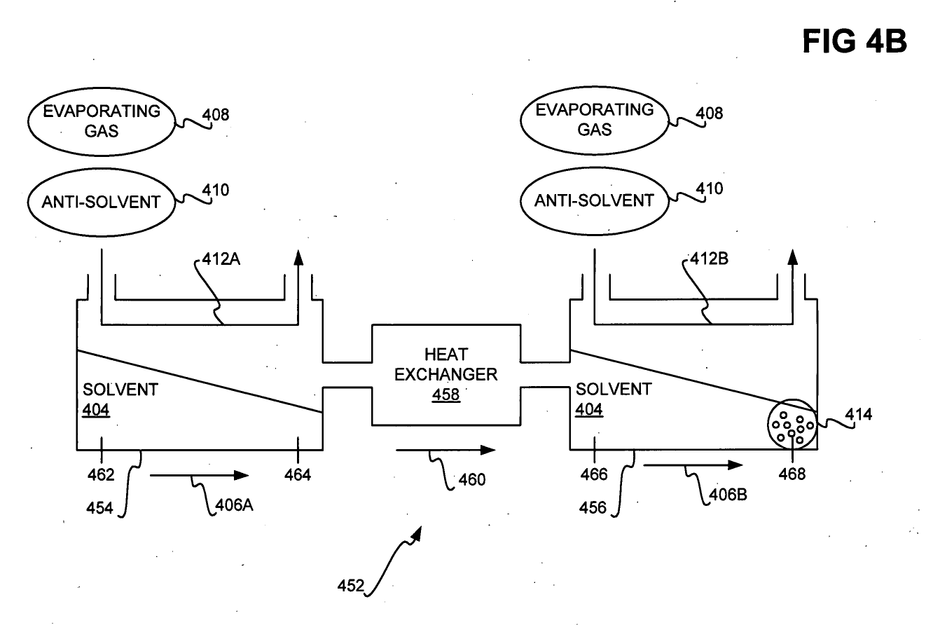

FIG. 4A illustratively depicts performance of parts 304, 306, and 308 of the method 300, according to an embodiment of the invention. A solvent 404, within which a pharmaceutical ingredient has been dissolved, is placed in a thin-film evaporation chamber 402, which is a single-stage chamber, at the left side of the chamber, which is not specifically depicted in FIG. 4A. The solvent 404 is in liquid form, and travels from left to right, as indicated by the arrow 406. The evaporation chamber 402 is a thin-film evaporation chamber in that there is a thin film of the solvent 404 at the bottom of the chamber 402.

An evaporating gas 408 and an anti-solvent 410 are introduced into the thin-film evaporation chamber 402 via an inlet port as shown in FIG. 4A. The evaporating gas 408 and the anti-solvent 410, which is in vapor form, pass over the solvent 404, as depicted by the arrow 412, before exiting the evaporation chamber 402 via an outlet port as shown in FIG. 4A. The passage of the evaporating gas 408 over the solvent 404 results in a cooling of the solvent 404, and a reduction of the volume of the solvent 404, such that there is less of the solvent 404 on the right-hand side of the chamber 402 as compared to the left-hand side of the chamber 402. Furthermore, the passage of the anti-solvent 410 over the solvent 404 results in condensation of the anti-solvent 410 within the solvent 404 in a homogeneous manner, which is not specifically depicted in FIG. 4A.

The evaporation of the solvent 404 due to passage of the evaporating gas 408 over the solvent 404, and the condensation of the anti-solvent 410 within the solvent 404 due to the passage of the anti-solvent 404 in vapor form over the solvent 404, result in the formation of a number of nanoparticles 414 of the pharmaceutical ingredient previously dissolved within the solvent 404. The nanoparticles 414 are thus formed at the right-hand side of the thin-film evaporation chamber 402. The solvent 404 at the right-hand side of the evaporation chamber 402, at which the nanoparticles 414 have been formed, is in the form of a slurry or a suspension. This mixture of the solvent 404 with the nanoparticles 414 can then exit the chamber 402 for any further processing that may be desired, and which is not specifically depicted in FIG. 4A.

Several points 416, 418, 420, and 422 are depicted in FIG. 4A along the thin-film evaporation chamber 402 in relation to which one particular example of nanoparticle formation within the evaporation chamber 402 is described. At the point 416, the relative humidity within the chamber 402 is approximately 60%, and the quantity of the gas within the evaporation chamber 402 that is evaporated solvent 404 is 0%. Furthermore, at the point 41.6, the temperature of the liquid solvent 404 is 25 degrees Celsius (° C.), and the liquid solvent 404 has a depth of 10 millimeters (mm). Also at the point 416, the mole fractions of the ethanol of the solvent 404, the chloroform of the solvent 404 (where the solvent 404 is ethanol:chloroform), and the anti-solvent 410 (where the anti-solvent 410 is specifically water) are 0.76, 0.24, and 0.00, respectively.

At the point 418, the relative humidity within the evaporation chamber 402 has decreased to 40%, and the quantity of the gas within the chamber 402 that is evaporated solvent 404 has risen to 40%. Furthermore, the temperature of the remaining liquid solvent 404 has dropped to 12° C., and the liquid solvent 404 just has a depth of 8 mm. The mole fractions of the ethanol, the chloroform, and the water are 0.72, 0.12, and 0.16, respectively, indicating precipitation of the anti-solvent 410 within the solvent 404.

Further along, at the point 420, the relative humidity within the evaporation chamber 402 has decreased to just 20%, and the quantity of the gas within the chamber 402 that is evaporated solvent 404 has risen even more to 75%. The temperature of the remaining liquid solvent 404 has dropped to 7° C., and the liquid solvent 404 now only has a depth of 4 mm. The mole fractions of the ethanol, the chloroform, and the water are 0.54, 0.02, and 0.44, respectively, indicating increased precipitation of the anti-solvent 410 within the solvent 404.

Finally, at the point 422, where formation of the nanoparticles 414 occurs, the relative humidity within the evaporation chamber 402 is now only 10%, and the quantity of the gas within the chamber 402 that is evaporated solvent 404 has risen to 90%. The temperature of the remaining liquid solvent 404 has dropped to 2° C., and the liquid solvent 404 has a depth of only 2 mm. The mole fractions of the ethanol, the chloroform, and the water are 0.20, 0.00, and 0.80, respectively, indicating that there is more precipitated anti-solvent 410 than solvent 404.

Referring back to FIG. 3, in one particular embodiment, the solvent may be further cooled by passing the solvent through a heat exchanger (310). For instance, the thin-film evaporation chamber may be a dual-stage chamber, instead of a single-stage chamber as depicted in FIG. 4A. Within a dual-stage evaporation chamber, evaporation of the solvent may be achieved in both stages of the chamber by passing an evaporating gas within both stages over the solvent. Similarly, within a dual-stage evaporation chamber, precipitation of the anti-solvent may be, but is not necessarily, achieved in both stages of the chamber by introducing the anti-solvent in vapor form within both stages over the solvent. In-between the stages of the evaporation chamber, the liquid solvent passes through the heat exchanger to further cool the solvent.

FIG. 4B illustratively depicts performance of parts 304, 306, 308, and 310 of the method 300, according to an embodiment of the invention. A dual-stage thin-film evaporation chamber 452 has a first stage 454 and a second stage 456, between which is fluidically connected a heat exchanger 458. The solvent 404, within which a pharmaceutical ingredient has been dissolved, is placed in the first stage 454 of the evaporation chamber 452. The solvent 404 is in liquid form, and travels from left to right within the first stage 454, as indicated by the arrow 406A.

The evaporating gas 408 and the anti-solvent 410 are introduced into the first stage 454 of the evaporation chamber 452 via an inlet port as shown in FIG. 4B. The evaporating gas 408 and the anti-solvent 410, which is in vapor form, pass over the solvent 404, as depicted by the arrow 412A, before exiting the first stage 454 via an outlet port as shown in FIG. 4B. The passage of the evaporating gas 408 over the solvent 404 cools the solvent 404 and reduces the volume of the solvent 404 via evaporation, such that there is less of the solvent 404 on the right-hand side of the stage 454 as compared to the left-hand side of the stage 454. Furthermore, the passage of the anti-solvent 410 over the solvent 404 can, but does not necessarily, result in precipitation of the anti-solvent 410 within the solvent 404 within the first stage 454 in a homogeneous manner, which is not specifically depicted in FIG. 4B.

The remaining liquid solvent 404 at the right-hand side of the first stage 454 of the evaporation chamber 452 then travels via tubing or piping to the heat exchanger 458, and then via tubing or piping to the left-hand side of the second stage 456 of the chamber 452, as indicated by the arrow 460. The heat exchanger 458 further cools the liquid solvent 404, which may also reduce the volume of the liquid solvent 404. Thus, the evaporation chamber 452 is a two-stage chamber in that it has two stages 454 and 456, which are connected to one another via the heat exchanger 458, which is present for further cooling of the solvent 404. It is noted that at least substantially none of the gas within the first stage 454 travels through the heat exchanger 458 to the second stage 456. For instance, at least substantially none of the evaporated solvent 404 travels from the first stage 454 to the second stage 456 via the heat exchanger 458.

Within the second stage 456 of the evaporation chamber 452, the solvent 404 travels from left to right, as indicated by the arrow 406B. The evaporating gas 408 and the anti-solvent 410 are also introduced into the second stage 456 via an inlet port as shown in FIG. 4B. The evaporating gas 408 and the anti-solvent 410, which is in vapor form, pass over the solvent 404, as depicted by the arrow 412B, before exiting the second stage 456 via an outlet port as shown in FIG. 4B. The passage of the evaporating gas 408 over the solvent 404 further cools the solvent and reduces the volume of the solvent 404 via evaporation, such that there is less of the solvent 404 on the right-hand side of the stage 456 as compared to the left-hand side of the stage 456. The passage of the anti-solvent 410 over the solvent 404 results in further precipitation of the anti-solvent 410 within the solvent 404 in a homogeneous manner, which is not specifically depicted in FIG. 4B.

The evaporation of the solvent 404 due to the passage of the evaporating gas 408 thereover, and the precipitation of the anti-solvent 410 within the solvent 404 due to the passage of the anti-solvent 410 thereover in vapor form, result in the formation of the nanoparticles 414 of the pharmaceutical ingredient previously dissolved within the solvent 404. The nanoparticles 414 are thus formed at the right-hand side of the second stage 456 of the thin-film evaporation chamber 452. The solvent 404 at the right-hand side of the second stage 456, at which the nanoparticles 414 have been formed, is in the form of a slurry or a suspension. This mixture of the solvent 404 with the nanoparticles 414 can then exit the second stage 456 of the evaporation chamber 452 for any further processing that may be desired, and which is not specifically depicted in FIG. 4B.

Several points 462, 464, 466, and 468 are depicted in FIG. 4B along the thin-film evaporation chamber 452, in relation to which one particular example of nanoparticle formation within the evaporation chamber 452 is described. At the point 462, the relative humidity within the stage 454 of the evaporation chamber 452 is 100%, and the quantity of the gas within the stage 454 that is evaporated solvent 404 is 0%. This is because the evaporating gas 408 and the anti-solvent 410 are introduced after the point 462. Furthermore, the temperature of the liquid solvent 404 at the point 462 is 45° C., and the liquid solvent 404 has a depth of 30 mm. Also at the point 462, the mole fractions of the ethanol of the solvent 404, the chloroform of the solvent 404 (where the solvent 404 is ethanol:chloroform), and the anti-solvent 410 (where the anti-solvent 410 is specifically water) are 0.80, 0.20, and 0.00, respectively.

At the point 464, the relative humidity within the stage 454 of the evaporation chamber 452 is now 60% and the quantity of gas within the stage 454 that is evaporated solvent 404 has increased to 40%, due to the introduction of the evaporating gas 408 and the anti-solvent 410. Furthermore, the temperature of the remaining liquid solvent 404 at the point 464 is 35° C., and the liquid solvent 404 has a depth of 10 mm. The mole fractions of the ethanol, the chloroform, and the water are 0.60, 0.20, and 0.20, respectively, indicating that some precipitation of the anti-solvent 410 has occurred within the solvent 404.

At the point 466, the relative humidity within the stage 456 of the evaporation chamber 452 is again 100%, and the quantity of gas within the stage 456 that is evaporated solvent 404 is 0%, since none of the gas of the first stage 454 of the chamber 452 passes to the second stage 456 via the heat exchanger 458. The temperature of the remaining liquid solvent 404 is 20° C., representing a temperature drop due to passage of the liquid solvent 404 through the heat exchanger 458, and the liquid solvent 404 has a depth of 10 mm, equal to its depth at the point 464 within the first stage 454. The mole fractions of the ethanol, the chloroform, and the water are still 0.60, 0.20, and 0.20, respectively, as they were at the point 464 within the first stage 454.

Finally, at the point 468, where formation of the nanoparticles 414 occurs, the relative humidity within the stage 456 of the evaporation chamber 452 is now only 60%, and the quantity of the gas within the stage 456 that is evaporated solvent 404 has risen to 40%. The temperature of the remaining liquid solvent 404 has dropped to 2° C., and the liquid solvent 404 has a depth of only 2 mm. The mole fractions of the ethanol, the chloroform, and the water are 0.20, 0.00, and 0.80, respectively, indicating that there is more precipitated anti-solvent 410 than solvent 404.

It is noted, therefore, that although specific embodiments have been illustrated and described herein, it will be appreciated by those of ordinary skill in the art that any arrangement calculated to achieve the same purpose may be substituted for the specific embodiments shown. This application is intended to cover any adaptations or variations of the disclosed embodiments of the present invention. It is thus manifestly intended that this invention be limited only by the claims and equivalents thereof.

We claim:

1. A method comprising:
dissolving a pharmaceutical ingredient within a solvent; and,
evaporating the solvent until a plurality of nanoparticles of the pharmaceutical ingredient are at least partially formed without employing a substrate for the nanoparticles,
such that a portion of the solvent remains within which the nanoparticles are located.

2. The method of claim 1, further comprising at least substantially reducing or stopping evaporation of the solvent to prevent degradation of the nanoparticles formed.

3. The method of claim 2, wherein at least substantially reducing or stopping evaporation of the solvent comprises adding an anti-solvent to the portion of the solvent remaining within which the pharmaceutical ingredient has been dissolved.

4. The method of claim 3, wherein the anti-solvent is one of water, an ethanol-water mixture, an alkane, an alkene, a cycloalkane, a supercritical fluid, or an ether.

5. The method of claim 2, wherein at least substantially reducing or stopping evaporation of the solvent comprises sealing the portion of the solvent remaining within which the pharmaceutical ingredient has been dissolved so that further evaporation cannot occur.

6. The method of claim 1, further comprising adding anti-solvent in vapor form such that the anti-solvent precipitates in relation to the solvent within which the pharmaceutical ingredient has been dissolved.

7. The method of claim 6, wherein addition of the anti-solvent in vapor form, along with evaporation of the solvent, results in formation of the nanoparticles.

8. The method of claim 6, wherein the anti-solvent is water.

9. The method of claim 1, further comprising at least one of cooling or reducing a volume of the solvent within which the pharmaceutical ingredient has been dissolved to promote formation of the nanoparticles.

10. The method of claim 1, wherein the pharmaceutical ingredient is one of: glyburide, prednisolone, and indomethacin, betamethasone acetate, triamcinolone acetonide, piroxicam, glimepiride, glipizide, or digoxin.

11. The method of claim 1, wherein the solvent is one of a single solvent or a multiple solvent.

12. The method of claim 1, wherein the solvent is a binary solvent, and the binary solvent is ethanol:chloroform.

13. The method of claim 12, wherein the ethanol is substantially 80% of the binary solvent by volume and the chloroform is substantially 20% of the binary solvent by volume.

14. The method of claim 1, wherein the solvent is a multiple solvent selected from: 80% methanol by volume and 20% water by volume; and, 80% acetone by volume and 20% water by volume.

15. A plurality of nanoparticles of a pharmaceutical ingredient formed by performing a method comprising:
evaporating a solvent within which the pharmaceutical ingredient has been dissolved until the nanoparticles of the pharmaceutical ingredient are formed, without employing a substrate for the nanoparticles; and,
at least substantially reducing or stopping evaporation of the solvent within which the nanoparticles have been formed to prevent degradation of the nanoparticles.

16. The nanoparticles of claim 15, wherein at least substantially reducing or stopping evaporation of the solvent comprises adding an anti-solvent to the solvent within which the nanoparticles have been formed.

17. The nanoparticles of claim 16, wherein the anti-solvent is one of water, an ethanol-water mixture, an alkane, an alkene, a cycloalkane, a supercritical fluid, or an ether.

18. The nanoparticles of claim 15, wherein at least substantially reducing or stopping evaporation of the solvent comprises sealing remaining of the solvent within which the nanoparticles have been formed.

19. The nanoparticles of claim 15, wherein the pharmaceutical ingredient is one of: glyburide, prednisolone, and indomethacin, betamethasone acetate, triamcinolone acetonide, piroxicam, glimepiride, glipizide, or digoxin.

20. The nanoparticles of claim 15, wherein the solvent is one of: a single solvent or a multiple solvent.

21. The nanoparticles of claim 15, wherein the solvent is a binary solvent, and the binary solvent is ethanol:chloroform.

22. The nanoparticles of claim 21, wherein the ethanol is substantially 80% of the binary solvent by volume and the chloroform is substantially 20% of the binary solvent by volume.

23. The method of claim 15, wherein the solvent is a multiple solvent selected from: 80% methanol by volume and 20% water by volume; and, 80% acetone by volume and 20% water by volume.

24. A plurality of nanoparticles of a pharmaceutical ingredient formed by performing a method comprising:
evaporating a solvent within which the pharmaceutical ingredient has been dissolved, resulting in a reduction in volume of the solvent and cooling of the solvent; and,
adding anti-solvent in vapor form such that the anti-solvent precipitates in relation to the solvent within which the pharmaceutical ingredient has been dissolved,
where evaporation of the solvent and addition of the anti-solvent results in formation of the nanoparticles without employing a substrate for the nanoparticles.

25. The nanoparticles of claim 24, wherein the anti-solvent is one of water, an ethanol-water mixture, an alkane, an alkene, a cycloalkane, a supercritical fluid, or an ether.

26. The nanoparticles of claim 24, the method further comprising placing the solvent within which the pharmaceutical ingredient has been dissolved within a thin-film evaporation chamber.

27. The nanoparticles of claim 26, wherein evaporating the solvent comprises passing an evaporating gas through the thin-film evaporation chamber.

28. The nanoparticles of claim 26, wherein adding the anti-solvent in vapor form comprises introducing the anti-solvent in vapor form within the thin-film evaporation chamber.

29. The nanoparticles of claim 26, wherein placing the solvent within the thin-film evaporation chamber comprises placing the solvent within a dual-stage thin-film evaporation chamber.

30. The nanoparticles of claim 29, wherein the method further comprises further cooling the solvent within which the pharmaceutical ingredient has been dissolved by passing the solvent through a heat exchanger.

31. The nanoparticles of claim 24, wherein the pharmaceutical ingredient is one of: glyburide, prednisolone, and indomethacin, betamethasone acetate, triamcinolone acetonide, piroxicam, glimepiride, glipizide, or digoxin.

32. The nanoparticles of claim 24, wherein the solvent is one of: a single solvent or a multiple solvent.

33. The nanoparticles of claim 32, wherein the solvent is a binary solvent, and the binary solvent is ethanol:chloroform, the ethanol being substantially 80% of the binary solvent by volume and the chloroform being substantially 20% of the binary solvent by volume.

34. The method of claim 24, wherein the solvent is a multiple solvent selected from: 80% methanol by volume and 20% water by volume; and, 80% acetone by volume and 20% water by volume.
